(19) United States
(12) Patent Application Publication
(10) Pub. No.: US 2026/0356775 A1
(43) Pub. Date: Nov. 12, 2026

(54) METHOD FOR PREPARING 1-ALKENES AND 1,2-ALKANEDIOLS

(71) Applicant: MINASOLVE SAS, Beuvry-La-Forêt (FR)

(72) Inventors: Markus NAHRWOLD, Mont-Saint-Guibert (BE); Arthur DUBOIS, Mont-Saint-Guibert (BE)

(21) Appl. No.: 19/488,728

(22) PCT Filed: May 31, 2024
(86) PCT No.: PCT/EP2024/065076

§ 371 Date: Nov. 28, 2025

(30) Foreign Application Priority Data
Jun. 1, 2023 (EP) 23176812.8

(51) Int. Cl. C07C 1/24; B01J 35/40; B01J 35/61

(52) U.S. Cl. CPC C07C 1/24; B01J 35/40; B01J 35/613; C07C 2521/06

(57) ABSTRACT

The current invention relates to a method for the preparation of 1,2-alkanediols from secondary alcohols, comprising the steps of: (a) dehydrating said secondary alcohols in the presence of a zirconia (ZrO₂) catalyst to produce primary alkenes; (b) oxidizing said primary alkenes to form 1,2-alkanediols, wherein the zirconia catalyst used in step (a) contains less than 0.2 wt. % silicon dioxide by weight of the catalyst and has a surface area of less than 100 m²/g.

### FIELD OF THE INVENTION

The invention relates to a process for the selective preparation of primary alkenes and vicinal 1,2-alkanediols from secondary alcohols of bio-based origin.

### BACKGROUND

Primary alkenes are important starting materials for many basic chemicals. A well-known example of alkene-derived ingredients for personal care products are 1,2-alkanediols. Vicinal 1,2-alkanediols are usually obtained by oxidation of 1-alkenes. The resulting diols are used as skin moisturizers, solvents and antimicrobial agents for cosmetics.

Nowadays, 1-alkenes are mainly produced by ZIEGLER oligomerization of ethylene. The ethylene is classically obtained by thermal cracking of hydrocarbons in the presence of steam. Ethylene is generally a fossil raw material from the oil industry, which makes it unsustainable. Other disadvantages of the ZIEGLER process are the high temperatures required, the complex product mixture that requires extensive purification, and the storage and handling of the gaseous, toxic and highly flammable intermediates. Furthermore, 1-alkenes obtained from the oligomerization of ethylene contain branched alkenes as major impurities. Their oxidation leads to undesired branched alkanediols, which are particularly difficult to separate from straight-chain 1,2-alkanediols.

Fossil carbon sources are generally classified as non-renewable and therefore considered unsustainable. Considering global attempts for achieving a sustainable economy, energy efficient processes using renewable raw materials can be regarded as technically advantageous over those using petrochemical feedstocks. Sustainable processes based on “green chemistry” generally have a lower negative impact on the environment. The term “green chemistry” also refers to more selective and energy-efficient processes. Such processes are hence commercially more attractive than conventional production processes.

A known pathway to synthesize 1-alkenes and 1,2-alkanediols from renewable raw materials starts with the dehydration of bio-based primary alcohols (US2019241491, WO2022122902). Dehydration of primary alcohols to 1-alkenes is typically carried out over an alumina catalyst at 255-380 °C. High selectivity for the primary alkene is achieved by working at low temperature at the expense of incomplete conversion (WO2022122902), by treating the catalyst with basic reagents (US2003/0065233, WO2019152569), or by chemically derivatising the catalyst (U.S. Pat. Nos. 9,242,226, 8,912,373), thereby inactivating acidic moieties due to trace amounts of silicon dioxide.

Primary biobased alcohols are usually obtained by hydrogenation of natural fatty acids or their corresponding esters. The C6-C12 fatty acids with an even number of carbon atoms are commonly isolated from palm kernel oil and coconut oil. The cultivation of oil and coconut palms requires a tropical climate, such as that found in tropical rainforests. Planting oil palms in these geographic areas reduces biodiversity. In addition, the subsequent use of abandoned oil palm plantations for food crops is problematic due to soil depletion. Another drawback is that short-chain fatty acids are only minor components of palm and coconut derived oils. Their isolation therefore requires extensive technical effort and natural resources. It is therefore desirable to find more sustainable alternatives to palm oil and coconut oil.

Another drawback of primary alcohols as raw material for 1-alkenes and 1,2-alkanediols is the high amount of energy needed for alcohol dehydration. It is well known that secondary alcohols are more readily dehydrated at lower temperatures than primary alcohols.

The main product of a dehydration of a secondary alcohol in the presence of an acidic catalyst is usually the thermodynamically more stable secondary alkene. Such conventional elimination reactions proceed via the E1 mechanism and follow the Saytzeff rule. In some cases, however, elimination of secondary alcohols occurs according to the Hofmann rule (E2 mechanism) and yields the primary alkene as the major product. One such case is the dehydration of secondary alcohols in the presence of zirconium dioxide (zirconia) catalysts, which provides primary alkenes as the main product. However, the selectivity is usually not complete, so that primary alkenes are always obtained alongside minor amounts of internal alkenes. Several examples to obtain primary alkenes by dehydration of secondary alcohols are described in the literature, such as EP0222356 and U.S. Pat. No. 5,210,363.

In summary, it would be desirable to have a sustainable raw material for the production of primary alkenes and 1,2-alkanediols. It should be possible to obtain this natural raw material without negative environmental impact. Potentially used arable land should subsequently be available again for food cultivation. Alternatively, the land used to produce the bio-based raw material should either not be suitable for food production or the raw material should be a by-product of food production.

### SUMMARY OF THE INVENTION

The present invention and embodiments thereof serve to provide a solution to one or more of above-mentioned disadvantages. To this end, the present invention relates to a process according to claim 1.

One objective of the present invention is to produce primary alkenes by dehydration of bio-based secondary alcohols. The primary alkene should be obtained in high yield with high conversion of the starting alcohol to the desired product. During this process, only minimal isomerisation of the double bond should occur. The process is preferably characterised by using a sufficient stream of inert gas to increase the selectivity for the primary alkene. The secondary alcohols as well as all corresponding downstream products according to this invention are preferably not derived from palm oil or palm kernel oil.

Another objective of the invention is the use of primary alkenes obtained from bio-based secondary alcohols as feedstock for the production of 1,2-alkanediols.

The present invention and embodiments thereof serve to provide a solution to one or more of the following problems:
Enabling the selective production of 1-alkenes from secondary alcohols by selective dehydration on a catalyst.
Decrease in the use of energy compared to the dehydration of primary alcohols.
Providing an alternative to palm kernel oil or coconut oil as feedstock for primary alkenes and corresponding 1,2-alkanediols, since bio-based secondary alcohols are commonly obtained from alternative sustainable feedstock such as castor oil, cellulose or hemicellulose.
Avoiding the formation of undesirable branched-chain alkenes and 1,2-alkanediols, which are difficult to remove by distillation from straight-chain primary alkenes and 1,2-alkanediols, respectively.

### DETAILED DESCRIPTION OF THE INVENTION

The present invention concerns a method for preparing 1-alkenes and 1,2-alkanediols from secondary alcohols.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In a first aspect, the invention provides a method for the preparation of 1,2-alkanediols from secondary alcohols.

In a preferred embodiment, the method comprises the step of:
a. dehydrating said secondary alcohols in the presence of a zirconia (ZrO₂) catalyst to produce primary alkenes.

The starting material used in the method of the present invention is a secondary alcohol. Preferably a secondary alcohol as represented by the formula (I),

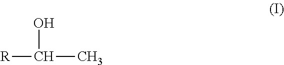

wherein R is a C1 to C30 straight chain, branched chain or cyclic hydrocarbon group. R is not particularly limited so far as it is a C1 to C20 straight chain, branched chain or cyclic hydrocarbon group, but it is preferably a C3 to C12 straight chain, branched chain or cyclic hydrocarbon group and more preferably a C3 to C12 straight chain hydrocarbon group. More preferably, R is a straight or branched hydrocarbon group. Preferred secondary alcohols are thus 2-alcohols. 2-alcohols are secondary alcohols wherein the hydroxyl group is attached to the second carbon atom of a hydrocarbon chain. Among the secondary alcohols represented by this formula (I), 3-methyl-2-butanol, 2-pentanol, 2-hexanol, 2-heptanol, 2-octanol, 2-nonanol, 2-decanol, 2-undecanol, 2-dodecanol, or combinations thereof are most preferred. In the practice of the present invention with this material, the OH group and the hydrogen atom of the methyl group in the formula (I) are released in the form of water to selectively produce primary alkene.

In a preferred embodiment, the secondary alcohols are chosen from the list of: 3-methyl-2-butanol, 2-pentanol, 2-hexanol, 2-heptanol, 2-octanol, 2-nonanol, 2-decanol, 2-undecanol, 2-dodecanol, or any combination thereof.

The secondary alcohols used as starting material for selective dehydration to primary alkenes are preferably bio-based, i.e., derived from a bio-sourced and/or non-petroleum-derived feedstock. The expression “bio-based” refers to materials from natural or renewable origin that are derived from living or organic matter that can be replenished over time, either naturally or through sustainable practices. This can include materials derived from renewable resources, such as plants, animals, or microorganisms.

Examples of natural and renewable origin include, but are not limited to, agricultural activities, fermentation of biomass, or the fixation of carbon dioxide from the air by means of technical measures such as the Fischer-Tropsch process. Such bio-based feedstocks therefore contain only modern carbon recently assimilated by a living organism or fixated from the surrounding atmosphere by a green chemical process. They stay in contrast to petrochemical or other fossil feedstock containing “old” carbon. Therefore, the term “sustainable” refers to materials that come from such renewable sources and are not derived from petrochemical or other fossil sources of carbon. The traditional method for testing the concentration of modern carbon in a product is to determine the level of carbon-14, a radioactive isotope of carbon that does not occur in fossil carbon. An alternative to radiocarbon analysis is the quantification of carbon-14 by Accelerated Mass Spectrometry (AMS).

In a preferred embodiment, the secondary alcohols are bio-based secondary alcohols. In a preferred embodiment, the secondary alcohols are derived from a renewable resource. In a further preferred embodiment, the renewable resource is selected from the list of: castor oil, cellulose, hemicellulose, or fermentation of bio-based feedstocks.

A preferred example of a suitable secondary alcohol is 2-octanol, which can be obtained by saponification of castor oil and subsequent pyrolysis. Castor oil is a vegetable oil obtained from the seeds of Ricinus communis and contains triglycerides of ricinoleic acid as its main component. The plant grows in poor soils, is resistant to drought and does not compete with the human food chain.

Other preferred secondary alcohols are bio-based 2-hexanol, preferably obtained from bio-based 2,5-dimethylfuran, and bio-based 2-pentanol, preferably derived from biobased 2-methylfuran. The aforementioned furans are commonly produced starting from natural cellulose or hemicellulose. Cellulose and hemicellulose are common waste products of food processing, especially of maize, rice and sugar from sugar cane or sugar beet. Therefore, no additional arable land is needed to provide these natural raw materials.

Alternatively, biobased secondary alcohols such as 2-pentanol and biobased 2-hexanol can be obtained from biobased carboxylic acids such as hexanoic and heptanoic acid, respectively. Short- and medium-chain carboxylic acids obtained by fermentation of bio-based feedstocks are commercially available, e.g. from AFYREN. Heptanoic acid (enanthic acid) is obtained from castor oil by pyrolysis of ricinoleic acid followed by oxidation. Nonanoic acid (pelargonic acid) is obtained by oxidative cleavage of oleic acid.

The dehydration reaction of the secondary alcohol is preferably carried out in the gas phase. Therefore, the secondary alcohol is vaporised prior to dehydration. The gaseous secondary alcohols are subsequently brought in the presence of the zirconia (zirconium oxide, ZrO₂) catalyst.

A preferred catalyst according to the invention minimises isomerisation, i.e., double bond isomerisation or skeletal isomerisation, which converts desirable non-isomerised 1-alkenes into undesirable isomerised higher alkenes. In particular, it was found that a minimized content of the impurity silicon dioxide (silica, SiO₂) decisively increases the selectivity of zirconia catalysts in the dehydration of secondary alcohols to primary alkenes without consecutive isomerisation of the double bond. Preferably, catalysts containing less than 0.2 wt. % of silica are used, more preferably catalysts containing less than 0.1 wt. % of silica, most preferably catalysts containing less than 0.05 wt. % of silica.

Treatment of the catalyst with bases, derivatising agents or other promoters has not been found to be mandatory, if a sufficiently pure grade of zirconia is used. However, surface treatment of the catalyst can be carried out optionally to further improve the selectivity for the primary alkene. Optional surface treatments include, for example, washing the catalyst with an alkaline liquid containing alkali or earth alkali salts or basic nitrogen species such as ammonia or amines. After the basic treatment, the solid catalyst can optionally be calcinated in the presence or absence of atmospheric oxygen.

Zirconium oxide used in the present invention is obtained by various methods, and particularly, one obtained by calcination of zirconium compounds at 300° to 1500 °C is preferred. In this case, preferred examples of zirconium compound used for calcination include for example zirconium hydroxide, zirconyl hydroxide, zirconium nitrate, zirconyl nitrate, zirconyl carbonate, zirconium alkoxide and the like. These compounds may be calcined in a state wherein they are supported on suitable carriers, or zirconium oxide after calcination may be supported on suitable carriers. Of course, it is also a preferred example of use to use zirconium oxide itself after calcination as a catalyst without using carriers. It is also possible to prepare the catalyst in the coexistence of a second component such as yttrium oxide, etc. if necessary.

The calcination is preferably carried out at temperatures below 1170 °C. In this way, a change in the crystal phase of the zirconia can be avoided. An alternative treatment of the catalyst is the capping of the acidic silica moieties by reaction with halo silanes such as trimethylchlorosilane.

Preferred zirconia catalysts according to the invention comprise primarily monoclinic zirconia. More preferably the catalyst comprises at least 80%, even more preferably at least 90% and most preferably at least 95% of zirconia in the monoclinic phase.

A further preferred catalyst according to the invention has a total surface area of less than 100 m²/g catalyst, preferably less than 90 m²/g, preferably less than 80 m²/g, preferably less than 70 m²/g, and even more preferably less than 60 m²/g.

Another or a further preferred catalyst has a total surface area of at least 10 m²/g, preferably at least 20 m²/g.

Another or a further preferred catalyst has a total surface area of between 10 and 100 m²/g, more preferably of between 20 and 80 m²/g, and most preferably of between 20 and 60 m²/g.

In a particularly preferred embodiment, the method comprises the step of:
a. dehydrating said secondary alcohols to produce primary alkenes, wherein the zirconia catalyst contains less than 0.2 wt. % silicon dioxide by weight of the catalyst and has a surface area of less than 100 m²/g, preferably less than 60 m²/g, or even less than 40 m²/g.

In a further or another particularly preferred embodiment, the method comprises the step of:
a. dehydrating said secondary alcohols to produce primary alkenes, wherein the zirconia catalyst contains less than 0.2 wt. % silicon dioxide by weight of the catalyst and has a surface area of between 10 and 100 m²/g, preferably of between 20 and 60 m²/g.

The catalyst may even have a total surface area of less than 50 m²/g catalyst, preferably less than 45 m²/g catalyst, more preferably less than 40 m²/g catalyst, even more preferably less than 35 m²/g catalyst, even more preferably a surface area of less than 30 m²/g catalyst, or even less than 25 m²/g catalyst.

Another or a further preferred catalyst has a total surface area of between 1 and 50 m2/g, more preferably of between 1 and 45 m²/g, more preferably of between 1 and 40 m²/g, even more preferably of between 1 and 35 m²/g catalyst, even more preferably of between 1 and 30 m²/g catalyst, or even of between 1 and 25 m²/g catalyst.

In a further or another particularly preferred embodiment, the method comprises the step of:
a. dehydrating said secondary alcohols to produce primary alkenes, wherein the zirconia catalyst contains less than 0.2 wt. % silicon dioxide by weight of the catalyst and has a surface area of between 1 and 45 m²/g, preferably of between 1 and 40 m²/g.

In some embodiments, the catalysts are porous ceramic bodies comprising zirconia. To this end the catalyst may be prepared by common processes, such as mixing zirconium hydroxide powder with a liquid and suitable additives (binders, extrusion agents, dispersants, further metal oxide) to form a mouldable mass, preparing discrete catalyst bodies from this mass, and sintering the bodies at a sufficient temperature for sufficient time to produce ceramic bodies. In this process, the primary crystalline phase of the zirconium is preferably monoclinic.

In a preferred embodiment, the catalyst has the form of pellets and/or spheres, preferably with an average diameter of between 0.1 mm and 10 mm, most preferably between 1 mm and 7 mm.

In a preferred embodiment, the catalyst is preheated prior to the dehydration reaction, preferably preheated under an inert carrier gas stream. In a further preferred embodiment, the catalyst is preheated to a temperature of 200 to 500 °C. Preferably, the catalyst is preheated to a temperature of between 200 to 400 °C, more preferably between 240 and 320 °C.

In a further preferred embodiment, the catalyst is preheated for between 5 and 60 minutes, preferably between 10 and 50 minutes, even more preferably between 20 and 40 minutes, and even more preferably between 25 and 35 minutes.

Optionally, the catalyst may contain binders, stabilizers or promotors that improve its selectivity, reactivity, physical stability (“crush strength”) or standing time. Preferred additives include metal oxides. Further preferred additives are selected from calcium oxide, cerium oxide, europium oxide, hafnium oxide, lanthanum oxide, magnesium oxide, tungsten oxide, yttrium oxide and ytterbium oxide.

In a further preferred embodiment, the reaction is carried out in the presence of a carrier gas, preferably an inert carrier gas. The inert carrier gas used in the present invention can be any gas that is non-reactive under the reaction conditions. Examples include nitrogen, helium, argon, water vapour or mixtures thereof. Preferably, nitrogen and/or argon are used as carrier gas, more preferably nitrogen is used as carrier gas.

In a preferred embodiment, the dehydrating reaction is carried out in the presence of an inert carrier gas with a flow rate of between 4 and 5000 liters of gas (measured at standard conditions) per liter of secondary alcohol used, preferably 4 and 2000 liters of gas (measured at standard conditions) per liter of secondary alcohol used, more preferably between 4 and 1000 liters of inert carrier gas per liter of secondary alcohol.

In a preferred embodiment, the dehydrating reaction is carried out in the presence of an inert carrier gas with a flow rate of at least 4 liters of gas (measured at standard conditions) per liter of secondary alcohol used, more preferably at least at least 5 liters of gas per liter of secondary alcohol used, more preferably at least at least 6 liters of gas per liter of secondary alcohol used, more preferably at least at least 7 liters of gas per liter of secondary alcohol used, more preferably at least at least 8 liters of gas per liter of secondary alcohol used, more preferably at least at least 9 liters of gas per liter of secondary alcohol used, more preferably at least at least 10 liters of gas per liter of secondary alcohol used, more preferably at least at least 11 liters of gas per liter of secondary alcohol used, more preferably at least at least 12 liters of gas per liter of secondary alcohol used, more preferably at least at least 13 liters of gas per liter of secondary alcohol used, more preferably at least at least 14 liters of gas per liter of secondary alcohol used, more preferably at least at least 15 liters of gas per liter of secondary alcohol used.

In a preferred embodiment, the dehydrating reaction is carried out in the presence of an inert carrier gas with a flow rate of between 4 and 5000 liters of gas (measured at standard conditions) per liter of secondary alcohol used, more preferably between 100 and 5000 liters of inert carrier gas per liter of secondary alcohol, more preferably between 200 and 5000 liters of inert carrier gas per liter of secondary alcohol, more preferably between 240 and 4800 liters of inert carrier gas per liter of secondary alcohol, or even between 240 and 3500 liters of inert carrier gas per liter of secondary alcohol or between 240 and 3000 liters of inert carrier gas per liter of secondary alcohol, or even between 1000 and 5000 liters of inert carrier gas per liter of secondary alcohol, or preferred between 2000 and 5000 liters of inert carrier gas per liter of secondary alcohol.

In a preferred embodiment, the dehydrating reaction is carried out in the presence of an inert carrier gas with a flow rate of at least 4 liters of gas (measured at standard conditions) per liter of secondary alcohol used, more preferably at least 50 liters of gas per liter of secondary alcohol used, more preferably at least 100 liters of gas per liter of secondary alcohol used, more preferably at least 150 liters of gas per liter of secondary alcohol used, more preferably at least 200 liters of gas per liter of secondary alcohol used, more preferably at least 220 liters of gas per liter of secondary alcohol used, more preferably at least 240 liters of gas per liter of secondary alcohol used, more preferably at least 500 liters of gas per liter of secondary alcohol used, more preferably at least 750 liters of gas per liter of secondary alcohol used, more preferably at least 800 liters of gas per liter of secondary alcohol used, more preferably at least 900 liters of gas per liter of secondary alcohol used, more preferably at least 1000 liters of gas per liter of secondary alcohol used, more preferably at least 1250 liters of gas per liter of secondary alcohol used, more preferably at least 1500 liters of gas per liter of secondary alcohol used, more preferably at least 1750 liters of gas per liter of secondary alcohol used, more preferably at least 2000 liters of gas per liter of secondary alcohol used, more preferably at least 2200 liters of gas per liter of secondary alcohol used.

In a preferred embodiment, the dehydrating reaction is carried out in the presence of an inert carrier gas with a flow rate of at most 10000 liters of gas (measured at standard conditions) per liter of secondary alcohol used, more preferably at most 5000 liters of gas per liter of secondary alcohol used, more preferably at most 4900 liters of gas per liter of secondary alcohol used, more preferably at most 4800 liters of gas per liter of secondary alcohol used, more preferably at most 4500 liters of gas per liter of secondary alcohol used, more preferably at most 4000 liters of gas per liter of secondary alcohol used, more preferably at most 3500 liters of gas per liter of secondary alcohol used, more preferably at most 3000 liters of gas per liter of secondary alcohol used.

The flow rate of the carrier gas is limited on one side to ensure a sufficient residence time of the starting material. This ensures a high conversion of the secondary alcohol to the alkene. At the same time, the carrier gas flow rate must be sufficiently high to achieve the maximum possible selectivity by preventing subsequent isomerization of the 1-alkene to higher alkenes. Finally, the residence time must be long enough for the system to reach equilibrium with a large proportion of primary alkene compared to higher alkenes. It was surprisingly found that a reduced residence time of the secondary alcohol and its dehydration products at the catalyst due to an increased stream of inert gas leads to a significantly higher selectivity for the primary alkene. This finding contradicts to prior art, where a change in the carrier gas stream was not identified as a decisive factor for achieving a higher selectivity. Furthermore, this is surprising as limiting the residence time in alternative ways did not consistently provide the same result. This is evidenced by decreasing the reactor size, which also reduces the residence time yet decreased rather than increased the selectivity for the primary alkene.

Surprisingly, it was also found that a minimum residence time is required to achieve the highest possible selectivity for the primary alkene. If the contact time is too short, the selectivity for the 1-alkene can decrease again.

In a further embodiment, the carrier gas is recirculated and reused. In this embodiment, the process may involve a gas/liquid separation and/or compression of the inert gas stream followed by reintroduction into the reactor.

Preferably, the secondary alcohol and the inert carrier gas are used without any additional solvent or diluent. However, within the scope of the invention, any solvent may be used in the dehydration reaction that does not adversely affect the reaction.

The reaction pressure is not particularly limited, and the reaction may be carried out at a reaction pressure of 0.01 bar to 50 bar. Preferably, the dehydration reaction is carried out at a pressure of between 0.1 bar and 25 bar, more preferably of between 0.1 bar and 10 bar, even more preferably of between 0.1 and 2 bar.

The method is particularly suitable for a continuous gas phase reaction. In this embodiment, the vaporised secondary alcohol and the inert carrier gas are passed continuously over a fixed or fluidized bed of catalyst, under the desired reaction conditions. Preferably, a tubular continuous reactor is used. Particularly preferably, a reactor with tube bundles is used. In this embodiment, the tubes may be heated to the desired temperature, e.g. by electrical heating elements or by a double jacket filled with thermal oil or molten salt. Further preferably a fluidized bed reactor is used, or even more preferred a continuous fluidized bed reactor is used.

Fluidized bed reactors are typically operated at higher flow rates than fixed bed reactors, and are therefore in some embodiments preferred, because it has been found, as described herein, that high flow rates are beneficial to the method.

In a particular preferred embodiment, a fluidized bed reactor is used in combination with high flow rates of an inert carrier gas, such as an inert carrier gas with a flow rate of at least 500, 1000 or even 1500 liters of gas (measured at standard conditions) per liter of secondary alcohol used. This combination allows upscaling the process to a continuous, industrial scale process while maintaining the conversion and selectivity obtained at lab scales. Reproducing the lab scale selectivity and conversion in a fixed bed reactor at industrial scale was found to be difficult. High inert carrier flow rates in a fixed bed reactor lead to channeling, i.e. preferred gas channels through the fixed catalyst bed resulting in maldistribution and reduced contact between gas species and catalyst, negatively impacting the conversion and/or selectivity.

The temperature of the dehydration reaction is chosen to ensure sufficient conversion and selectivity and to avoid the formation of the corresponding ketones and/or ethers as undesirable dehydration by-products. In a preferred embodiment, temperatures in the range of about 200 °C to 500 °C are suitable to facilitate the desired dehydration reaction. Preferably, the dehydration reaction is carried out at temperatures in the range of 200 °C to 400 °C, more preferably between 240 °C and 350 °C, more preferably between 240 °C and 320 °C, more preferably between 240 °C and 320 °C, more preferably between 240 °C and 300 °C, more preferably between 240 °C and 280 °C, most preferably between 240 °C and 260 °C.

In a preferred embodiment, the temperature of the dehydration reaction is at least 200 °C, more preferably at least 210 °C, more preferably at least 220 °C, more preferably at least 230 °C, more preferably at least 240 °C, more preferably at least 250 °C. Increasing the temperature increases the conversion of the secondary alcohol.

In a preferred embodiment, the temperature of the dehydration reaction is at most 400 °C, more preferably at most 380 °C, more preferably at most 360 °C, more preferably at most 350 °C, more preferably at most 340 °C, more preferably at most 330 °C, more preferably at most 320 °C, more preferably at most 310 °C, more preferably at most 300 °C. The inventors found that increasing temperature increases the conversion, but decreases the selectivity of the primary alkene. Sufficient selectivity for the primary alkene is highly desirable as it allows higher yields and more importantly easier separation of the products for the oxidation to 1,2-alkanedoils.

In a particularly preferred embodiment, the temperature of the dehydration reaction is between 240 and 350 °C and is carried out in the presence of an inert carrier gas with a flow rate of between 10 and 5000, or between 10 and 2000 liters of gas (measured at standard conditions) per liter of secondary alcohol used, more preferably between 150 and 5000, or between 240 and 5000 liters of inert carrier gas per liter of secondary alcohol. In a particular preferred embodiment, the dehydration reaction is carried out between 240 and 350 °C, in the presence of an inert carrier gas with a flow rate between 1500 and 5000 liters of inert carrier gas per liter of secondary alcohol used. The inventors surprisingly found this combination allows for high conversion of the secondary alcohol with a high selectivity for the primary alkene. Furthermore, when starting from straight chain secondary alcohols, the desired product as well as the byproducts are straight chain alkene isomers. These reaction conditions avoid the formation of branched vinylic alkenes (almost) entirely. This is particularly desirable as it allows for easier separation of the product streams. Furthermore, the reaction is operated at reasonably low temperatures, allowing improved energy efficiency in particular when recuperating reaction heat.

The secondary alcohol used as starting material according to the invention is preferably fed to the gas phase reactor at a rate suitable to allow an optimum balance of maximum conversion and maximum selectivity. The feed rate is calculated as the Liquid Hourly Space Velocity (LHSV), which is the volume of starting material injected per hour per reactor volume, expressed as “1/h” or “h⁻¹”.

LHSV=(volume of secondary alcohol)/(volume of reactor·hour)

Preferably, the feed rate of the secondary alcohol is selected from a LHSV of between 0.1 and 10 h⁻¹, more preferably between 0.2 and 8 h⁻¹, even more preferably between 0.3 and 6 h⁻¹, even more preferably between 0.4 and 4 h⁻¹, most preferably between 0.5 and 3.5 h⁻¹.

The product mixture resulting from step (a) is optionally separated into desired 1-alkenes, by-products and unreacted starting materials by conventional methods such as solvent extraction, phase separation, fractional distillation, etc. A particularly suitable method for purifying the desired 1-alkene is to separate the phases of the crude product and the water formed during the dehydration reaction, followed by fractional distillation of the alkene phase. In a preferred embodiment, the method comprises a distillation step between the dehydration and oxidation steps.

In a particularly preferred embodiment, the dehydration reaction is carried out using a straight chain secondary alcohol, at conditions which favour the formation of primary alkenes and smaller amounts of secondary and higher alkenes; while avoiding the formation of branched vinylic alkenes. Preferably the amount of primary alkene is at least 80 wt. %, more preferably at least 85 wt. %, more preferably at least 90 wt. %, more preferably at least 91 wt. %, more preferably at least 92 wt. %, more preferably at least 93 wt. %, more preferably at least 94 wt. %, more preferably at least 95 wt. %, more preferably at least 96 wt. %, more preferably at least 97 wt. %, more preferably at least 98 wt. %, more preferably at least 99 wt. %. The inventors found that a mixture of at least 80 wt. % straight primary alkenes and smaller amounts of straight secondary and straight higher alkenes can be integrally oxidized; after which the vicinal alkanediols can be separated from the by-products. This significantly limits the purification requirements as A) no difficult to separate branched vinylic alkenes are formed in the dehydration step, reducing the amount of by-products subsequent to oxidation and simplifying the purification and B) the intermediate purification requirements are significantly lower as sufficiently high purity of the alkene phase is obtained from the reaction thus no purification or separation of primary alkenes from secondary and higher alkenes is required.

The provided secondary alcohols are dehydrated to give mainly the corresponding primary alkene and smaller amounts of secondary and higher alkenes. The term “corresponding” refers to the alkenes which are produced by the method of the invention and correspond in the length of their carbon chain to the length of the carbon chain of the starting secondary alcohol.

Potentially unreacted secondary alcohol is preferably reused and subjected to a further dehydration reaction, either with or without prior purification. More preferably, this process of recycling the starting material is carried out in a continuous or semi-continuous manner.

In a further particularly preferred embodiment, the method comprises further the step of:
b. oxidizing said primary alkenes to form 1,2-alkanediols.

The obtained primary alkenes (1-alkenes) are typically free of branched isomers. This makes them particularly useful for conversion by oxidation to the corresponding 1,2-alkanediols, preferably vicinal 1,2-alkanediols. These diols are commonly used as solvents, humectants, conditioners, rheology modifiers and antimicrobial components. They are applied as additives in compositions utilized in various industries, including personal care, cleaning, and medical applications. The most commonly used 1,2-alkanediols contain a straight chain of 3 to 30 carbon atoms. Well-known examples include 1,2-propanediol (propylene glycol), 1,2-butanediol, 1,2-pentanediol (pentylene glycol), 1,2-hexanediol, 1,2-heptanediol, 1,2-octanediol (caprylyl glycol), 1,2-decanediol (decylene glycol) and 1,2-dodecanediol (lauryl glycol). Longer chain 1,2-alkanediols comprise, for example, 1,2-tetradecanediol (myristyl glycol), 1,2-octacosanediol (octacosanyl glycol) and mixtures such as C14-C18 glycol, C18-C30 glycol and C20-C30 glycol.

The most common synthesis route for producing such 1,2-alkanediols involves the oxidation of 1-alkenes. Preferably with aqueous hydrogen peroxide as a reagent in combination with a carboxylic acid such as formic acid or acetic acid. A corresponding peracid is formed in situ, which reacts with the double bond of the alkene. Additional catalysts may be added to the reaction mixture to improve the yield and selectivity of the oxidation reaction. The resulting epoxy alkanes are reacted with water to give the alkanediols, together with the corresponding formate or acetate esters. The obtained mixture of products is commonly contacted with a suitable base, such as aqueous sodium hydroxide or potassium hydroxide, to convert the alkanediol esters to the free alkanediol. The final 1,2-alkanediol is separated from the by-products by means of fractional distillation.

It was surprisingly found that even larger amounts of linear alkanediol isomers (2,3-alkanediols, 3,4-alkanediols, . . . ) can be smoothly separated from the desired 1,2-alkanediols by fractional distillation. In order to ensure a high yield of 1,2-alkanediol, the crude mixture of alkanediols should comprise 1,2-alkanediol as the major alkanediol isomer. At least 50% of all alkanediols in the crude product is preferably 1,2-alkanediol, more preferably at least 80%, most preferably at least 90%.

The process according to the invention is also characterised by the fact that virtually no branched vinylic alkenes are produced when starting from straight chain secondary alcohols. This simplifies the purification of the corresponding 1,2-alkanediols, which are typically easier to separate by distillation from straight-chain isomers than from branched isomers.

In a particularly preferred embodiment, the method for the preparation of 1,2-alkanediols from secondary alcohols, comprises the steps of:
a. dehydrating said secondary alcohols in the presence of a zirconia (ZrO2) catalyst to produce primary alkenes;
b. oxidizing said primary alkenes to form 1,2-alkanediols, characterized in that, the zirconia catalyst used in step (a) contains less than 0.2 wt. % silicon dioxide by weight of the catalyst and has a surface area of less than 40 m²/g, wherein the dehydrating is carried out in the presence of an inert carrier gas with a flow rate of between 150 and 5000 liters of gas (measured at standard conditions) per liter of secondary alcohol used, and wherein the dehydrating is carried out in a fluidized bed reactor.

The following are embodiments of the invention:
1. A method for the preparation of 1,2-alkanediols from secondary alcohols, comprising the steps of:
   a. dehydrating said secondary alcohols in the presence of a zirconia (ZrO₂) catalyst to produce primary alkenes;
   b. oxidizing said primary alkenes to form 1,2-alkanediols, characterized in that, the zirconia catalyst used in step (a) contains less than 0.2 wt. % silicon dioxide by weight of the catalyst and has a surface area of less than 100 m²/g.
2. The method according to embodiment 1, wherein the secondary alcohols are derived from bio-based secondary alcohol derived from a renewable resource.
3. The method according to embodiment 2, wherein the renewable resource is selected from the list of: castor oil, cellulose or hemicellulose.
4. The method according to any of the previous embodiments, wherein the dehydrating is carried out in the presence of an inert carrier gas with a flow rate of between 4 and 2000 liters of gas (measured at standard conditions) per liter of secondary alcohol used, more preferably between 4 and 20 liters of inert carrier gas per liter of secondary alcohol.
5. The method according to any of the previous embodiments, wherein the secondary alcohols are straight chain, branched chain or cyclic secondary alcohols having from 2 to 30 carbon atoms.
6. The method according to any of the previous embodiments, wherein the secondary alcohols are chosen from the list of: 3 methyl-2-butanol, 2-pentanol, 2-hexanol, 2-heptanol, 2-octanol, 2-nonanol, 2-decanol, 2-undecanol, 2-dodecanol, or any combination thereof.
7. The method according to any of the previous embodiments, wherein the zirconia catalyst is present in the form of pellets and/or spheres with an average diameter of between 0.1 mm and 10 mm, preferably between 1 mm and 7 mm.
8. The method according to any of the previous embodiments, wherein the zirconia catalyst contains at least 80%, even more preferably at least 90% and most preferably at least 95% of zirconia in the monoclinic phase.
9. The method according to any of the previous embodiments, wherein the dehydration is carried out as a continuous gas phase reaction.
10. The method according to embodiment 9, wherein secondary alcohols are fed to the reactor with a liquid hourly space velocity (LHSV) of between 0.1 and 10 h⁻¹, preferably between 0.5 and 3.5 h⁻¹.
11. The method according any of the previous embodiments, wherein the zirconium dioxide catalyst has a surface area of less than 60 m²/g.
12. The method according to any of the previous claims, wherein the dehydration step is carried out at a temperature between 200 °C and 500 °C.
13. The method according to any of the previous embodiments, wherein the catalyst is preheated at a temperature between 200 and 400 °C under an inert gas stream.
14. The method according to any of the previous embodiments, further comprising a distillation step between the dehydration and oxidation steps.
15. Primary alkenes obtained through a method according to any of embodiments 1-14.
16. 1,2-alkanediols obtained through a method according to any of embodiments 1-14.

Although the first aspect of the invention is focused on the preparation of 1,2-alkanediols, it is obvious that the first step of the method according to the first aspect, i.e. dehydrating secondary alcohols to primary alkenes is also subject of the invention. Therefore, the invention provides in a second aspect a method for the preparation of primary alkenes from secondary alcohols. All of the above embodiments are also applicable to this aspect of the invention.

The present invention will be now described in more details, referring to examples that are not limitative.

### EXAMPLES

### General Procedure for Dehydration Reactions:

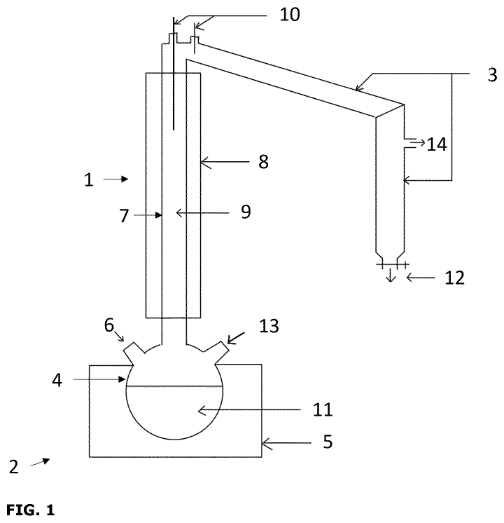

The apparatus used for the dehydration reactions is shown in FIG. 1. It consists of a heated fixed bed tube reactor 1, an evaporator 2, and a condenser 3.

A flask 4 equipped with an electric heating jacket 5 and an inert gas inlet 6 is used as evaporator 2 for the secondary alcohol 11. The flask 4 is further connected to a vertically mounted borosilicate glass tube 7 of 1 cm diameter and 30 cm length (volume: 23.6 mL). The tube 7 is surrounded by an electric heating jacket 8 and filled with solid catalyst 9 (extrudate, 3 mm in diameter and 3-15 mm in length). The temperature of the catalyst 9 in the reactor 1 is monitored by a type-K temperature probe 10. The upper end of the reactor tube is connected to a condenser 3.

The secondary alcohol 11 is continuously fed by a syringe pump 13 into the evaporation flask 4 and is evaporated under atmospheric pressure by heating above its boiling point. The vapor is taken up by a constant stream of nitrogen and transferred to the heated tube 7 containing the catalyst 9. The product mixture 12 obtained is liquefied at the condenser 3 and collected in portions of between 1 ml and 20 mL. The inert gas leaves the condenser at an inert gas outlet 14.

The conversion of the secondary alcohol and the selectivity for the different products obtained are determined by GC-FID. Conversion and selectivity are calculated as follows:

Conversion of 2-alcohol=100%−(area-% of unreacted secondary alcohol)·

Selectivity for 1-alkene=(area-% of 1-alkene)/(sum of area-% of all-products)

### Examples 1-27

Following the general procedure for dehydration reactions, bio-based 2-octanol derived from pyrolysis and saponification of castor oil was reacted on a zirconium dioxide catalyst containing less than 0.2% silicon dioxide and characterized by a surface area of 58 m²/g. The results obtained at different temperatures, nitrogen flow rates and 2-octanol feed rates are summarized in Table 2.

TABLE 2

| | | | | | Products | |
|---|---|---|---|---|---|---|
| | Temp. | | | | | Selectivity |
| | of | | 2-octanol | | Conversion | for |
| | Catalyst | N₂ flow | Feed rate | LHSV | of 2-octanol | 1-octene |
| # | (°C) | (L/min) | (mL/h) | (1/h) | (%) | (%) |
| 1 | 298 | 1.0 | 50 | 2.1 | 91 | 88 |
| 2 | 285 | 1.0 | 50 | 2.1 | 81 | 89 |
| 3 | 258 | 1.0 | 50 | 2.1 | 73 | 90 |
| 4 | 312 | 0.7 | 50 | 2.1 | 94 | 84 |
| 5 | 295 | 0.7 | 50 | 2.1 | 93 | 86 |
| 6 | 283 | 0.7 | 50 | 2.1 | 89 | 88 |
| 7 | 262 | 0.7 | 50 | 2.1 | 87 | 89 |
| 8 | 311 | 0.5 | 50 | 2.1 | 97 | 79 |
| 9 | 284 | 0.5 | 50 | 2.1 | 98 | 81 |
| 10 | 252 | 0.5 | 50 | 2.1 | 88 | 88 |
| 11 | 288 | 0.4 | 50 | 2.1 | 98 | 81 |
| 12 | 253 | 0.4 | 50 | 2.1 | 94 | 85 |
| 13 | 245 | 0.4 | 50 | 2.1 | 90 | 87 |
| 14 | 290 | 0.3 | 50 | 2.1 | 100 | 77 |
| 15 | 281 | 0.3 | 50 | 2.1 | 98 | 81 |
| 16 | 242 | 0.3 | 50 | 2.1 | 94 | 85 |
| 17 | 291 | 0.2 | 50 | 2.1 | 100 | 72 |
| 18 | 272 | 0.2 | 50 | 2.1 | 100 | 77 |
| 19 | 303 | 1.0 | 25 | 1.1 | 96 | 81 |
| 20 | 292 | 1.0 | 25 | 1.1 | 89 | 86 |
| 21 | 283 | 1.0 | 25 | 1.1 | 73 | 89 |
| 22 | 269 | 1.0 | 25 | 1.1 | 56 | 91 |
| 23 | 291 | 0.7 | 25 | 1.1 | 94 | 86 |
| 24 | 278 | 0.7 | 25 | 1.1 | 83 | 88 |
| 25 | 267 | 0.7 | 25 | 1.1 | 64 | 91 |
| 26 | 294 | 0.5 | 25 | 1.1 | 97 | 83 |
| 27 | 285 | 0.5 | 25 | 1.1 | 96 | 86 |

### Examples 28-30

The reaction conditions of examples 1-27 were used, but the catalyst was preheated for 30 min at 320 °C under a nitrogen stream. The results obtained are summarized in Table 3.

TABLE 3

| | | | | | Products | |
|---|---|---|---|---|---|---|
| | | | 2-octanol | | | Selectivity |
| | Temp. of | | Feed | | Conversion | for |
| | Catalyst | N₂ flow | rate | LHSV | of 2-octanol | 1-octene |
| # | (°C) | (L/min) | (mL/h) | (1/h) | (%) | (%) |
| 28 | 323 | 0.7 | 50 | 2.1 | 98 | 77 |
| 29 | 294 | 0.7 | 50 | 2.1 | 97 | 89 |
| 30 | 280 | 0.7 | 50 | 2.1 | 91 | 91 |

The preheated catalyst gave a cleaner reaction and higher conversion and selectivity than the non-preheated catalyst (see examples 4-6 for comparison).

### Examples 31-36

The reaction conditions of examples 1-27 were used, but the height of the tube reactor was only 15 cm instead of 30 cm. The results obtained are summarized in Table 4.

TABLE 4

| | | | | | Products | |
|---|---|---|---|---|---|---|
| | Temp. | | | | | Selectivity |
| | of | | 2-octanol | | Conversion | for |
| | Catalyst | N₂ flow | Feed rate | LHSV | of 2-octanol | 1-octene |
| # | (°C) | (L/min) | (mL/h) | (1/h) | (%) | (%) |
| 31 | 325 | 0.7 | 50 | 4.2 | 99% | 56% |
| 32 | 309 | 0.7 | 50 | 4.2 | 98% | 74% |
| 33 | 287 | 0.7 | 50 | 4.2 | 96% | 85% |
| 34 | 282 | 0.7 | 25 | 2.1 | 99% | 60% |
| 35 | 275 | 0.7 | 25 | 2.1 | 98% | 69% |
| 36 | 264 | 0.7 | 25 | 2.1 | 98% | 74% |

The observed selectivity for the product 1-octene was significantly lower when the length of the tube reactor was decreased by 50% (see examples 4-6 and 23-25 for comparison).

### Examples 37-46

Following the general procedure for dehydration reactions, bio-based 2-octanol was reacted on a zirconium dioxide catalyst containing less than 0.2% silicon dioxide and characterized by a surface area of 29 m²/g. The results obtained are summarized in Table 5.

TABLE 5

| | | | | | Products | |
|---|---|---|---|---|---|---|
| | Temp. | | 2-octanol | | | Selectivity |
| | of | | Feed | | Conversion | for |
| | Catalyst | N₂ flow | rate | LHSV | of 2-octanol | 1-octene |
| # | (°C) | (L/min) | (mL/h) | (1/h) | (%) | (%) |
| 37 | 251 | 0.7 | 50 | 2.1 | 96% | 92% |
| 38 | 271 | 0.7 | 50 | 2.1 | 96% | 89% |
| 39 | 304 | 0.7 | 50 | 2.1 | 97% | 82% |
| 40 | 321 | 0.7 | 50 | 1.1 | 98% | 69% |
| 41 | 245 | 0.4 | 50 | 1.1 | 96% | 91% |
| 42 | 287 | 0.4 | 50 | 1.1 | 96% | 86% |
| 43 | 311 | 0.4 | 50 | 1.1 | 99% | 66% |
| 44 | 244 | 0.2 | 50 | 1.1 | 95% | 87% |
| 45 | 288 | 0.2 | 50 | 1.1 | 99% | 65% |
| 46 | 308 | 0.2 | 50 | 1.1 | >99% | 39% |

### Comparative Examples 47-52

Following the general procedure for dehydration reactions, bio-based 2-octanol was reacted on a zirconium dioxide catalyst containing 40% titanium dioxide and characterized by a surface area of 84 m²/g.

The results obtained at different temperatures and 2-octanol feed rates are summarized in Table 6.

TABLE 6

| | | | | | Products | |
|---|---|---|---|---|---|---|
| | | | 2-octanol | | | Selectivity |
| | Temp. of | | Feed | | Conversion | for |
| | Catalyst | N₂ flow | rate | LHSV | of 2-octanol | 1-octene |
| # | (°C) | (L/min) | (mL/h) | (1/h) | (%) | (%) |
| 47 | 339 | 0.8 | 50 | 2.1 | 100 | 10 |
| 48 | 308 | 0.8 | 50 | 2.1 | 100 | 13 |
| 49 | 286 | 0.8 | 50 | 2.1 | 100 | 20 |
| 50 | 307 | 0.5 | 50 | 2.1 | 100 | 6 |
| 51 | 290 | 0.5 | 50 | 2.1 | 100 | 11 |
| 52 | 277 | 0.5 | 50 | 2.1 | 100 | 14 |

Compared to the pure zirconia catalyst according to a preferred embodiment of the invention, the mixed oxide catalyst containing 40% titanium dioxide gave a higher conversion, but with a much lower selectivity for the primary alkene.

### Comparative Examples 53-58

Following the general procedure for dehydration reactions, bio-based 2-octanol was reacted on a zirconium dioxide catalyst containing less than 0.2% silicon dioxide and characterized by a surface area of 102 m²/g.

The results obtained at different temperatures, nitrogen flow rates and 2-octanol feed rates are summarized in Table 7.

TABLE 7

| | | | | | Products | |
|---|---|---|---|---|---|---|
| | | | 2-octanol | | | Selectivity |
| | Temp. of | | Feed | | Conversion | for |
| | Catalyst | N₂ flow | rate | LHSV | of 2-octanol | 1-octene |
| # | (°C) | (L/min) | (mL/h) | (1/h) | (%) | (%) |
| 53 | 339 | 1.0 | 230 | 9.7 | 45 | 67 |
| 54 | 308 | 1.0 | 139 | 5.9 | 44 | 67 |
| 55 | 286 | 1.0 | 52 | 2.2 | 98 | 61 |
| 56 | 307 | 1.0 | 40 | 1.7 | 97 | 67 |
| 57 | 290 | 1.0 | 25 | 1.1 | 99 | 65 |
| 58 | 277 | 1.0 | 7 | 0.3 | 39 | 66 |

The zirconia catalyst with a surface area of 102 m²/g showed a lower selectivity compared to the zirconia catalyst according to a preferred embodiment of the invention, having a surface area of less than 100 m²/g.

### Examples 59-67

Following the general procedure for dehydration reactions, bio-based 2-octanol was reacted on a zirconium dioxide catalyst containing less than 0.2% silicon dioxide and characterized by a surface area of 100 m²/g. The catalyst beads had a spherical shape with a diameter of 1.6 mm. The reactor was operated with high carrier gas flow rates under fluidised bed conditions.

The results obtained at different temperatures, nitrogen flow rates and 2-octanol feed rates are summarized in Table 8.

TABLE 8

| | | | | | Products | |
|---|---|---|---|---|---|---|
| | | | 2-octanol | | | Selectivity |
| | Temp. of | | Feed | | Conversion | for |
| | Catalyst | N₂ flow | rate | LHSV | of 2-octanol | 1-octene |
| # | (°C) | (L/min) | (mL/h) | (1/h) | (%) | (%) |
| 59 | 313 | 2.0 | 50 | 2.1 | 68 | 94 |
| 60 | 355 | 2.0 | 50 | 2.1 | 98 | 91 |
| 61 | 423 | 2.0 | 50 | 2.1 | 100 | 62 |
| 62 | 309 | 3.0 | 50 | 2.1 | 47 | 94 |
| 63 | 351 | 3.0 | 50 | 2.1 | 97 | 91 |
| 64 | 411 | 3.0 | 50 | 2.1 | 100 | 69 |
| 65 | 304 | 4.0 | 50 | 2.1 | 34 | 95 |
| 66 | 346 | 4.0 | 50 | 2.1 | 64 | 91 |
| 67 | 403 | 4.0 | 50 | 2.1 | 97 | 80 |

### Examples 68-75

Following the general procedure for dehydration reactions, bio-based 2-octanol was reacted on a zirconium dioxide catalyst containing less than 0.2% silicon dioxide and characterized by a surface area of 26 m²/g. The catalyst beads had a mean diameter of 1.5 mm. The reactor was operated with high carrier gas flow rates under fluidised bed conditions.

The results obtained at different temperatures, nitrogen flow rates and 2-octanol feed rates are summarized in Table 9.

TABLE 9

| | | | | | Products | |
|---|---|---|---|---|---|---|
| | | | 2-octanol | | | Selectivity |
| | Temp. of | | Feed | | Conversion | for |
| | Catalyst | N₂ flow | rate | LHSV | of 2-octanol | 1-octene |
| # | (°C) | (L/min) | (mL/h) | (1/h) | (%) | (%) |
| 68 | 323 | 2.0 | 50 | 2.1 | 83 | 91 |
| 69 | 351 | 2.0 | 50 | 2.1 | 98 | 89 |
| 70 | 424 | 2.0 | 50 | 2.1 | 100 | 78 |
| 71 | 331 | 3.0 | 50 | 2.1 | 70 | 91 |
| 72 | 358 | 3.0 | 50 | 2.1 | 91 | 89 |
| 73 | 422 | 3.0 | 50 | 2.1 | 100 | 83 |
| 74 | 359 | 4.0 | 50 | 2.1 | 86 | 89 |
| 75 | 427 | 4.0 | 50 | 2.1 | 100 | 83 |

Interestingly, under fluidized bed conditions the surface area of the catalyst is less decisive. The catalyst in examples 59-67 (100 m²/g) is even more selective than the ones in examples 68-75 (26 m²/g) or examples 76-84 (16 m²/g). Also, the reaction temperature can be lowered in case of higher surface catalysts.

### Examples 76-84

Following the general procedure for dehydration reactions, bio-based 2-octanol was reacted on a zirconium dioxide catalyst containing less than 0.2% silicon dioxide and characterized by a surface area of 16 m²/g. The catalyst beads had a mean diameter of 1.5 mm. The reactor was operated with high carrier gas flow rates under fluidised bed conditions.

The results obtained at different temperatures, nitrogen flow rates and 2-octanol feed rates are summarized in Table 10.

TABLE 10

| | | | | | Products | |
|---|---|---|---|---|---|---|
| | | | 2-octanol | | | Selectivity |
| | Temp. of | | Feed | | Conversion | for |
| | Catalyst | N₂ flow | rate | LHSV | of 2-octanol | 1-octene |
| # | (°C) | (L/min) | (mL/h) | (1/h) | (%) | (%) |
| 76 | 325 | 2.0 | 50 | 2.1 | 82 | 92 |
| 77 | 350 | 2.0 | 50 | 2.1 | 97 | 88 |
| 78 | 418 | 2.0 | 50 | 2.1 | 99 | 70 |
| 79 | 327 | 3.0 | 50 | 2.1 | 68 | 92 |
| 80 | 356 | 3.0 | 50 | 2.1 | 92 | 90 |
| 81 | 418 | 3.0 | 50 | 2.1 | 100 | 81 |
| 82 | 333 | 4.0 | 50 | 2.1 | 45 | 92 |
| 83 | 358 | 4.0 | 50 | 2.1 | 84 | 87 |
| 84 | 422 | 4.0 | 50 | 2.1 | 100 | 82 |

### Example 85

The crude 1-octene obtained from the dehydration of bio-based 2-octanol was separated by distillation from the remaining starting material and the higher boiling by-products. The distillation was carried out at ambient pressure and the distillate was collected in 20 ml fractions. The results are summarized in Table 11. Fractions 1-9 containing 1-octene with a purity of at least 85 area % (GC-FID) were selected for conversion in the subsequent oxidation step.

TABLE 11
Area percent (%)

| | | 2-octene | 3-octene | 2- | 2- |
|---|---|---|---|---|---|
| | 1-octene | (cis/trans) | (cis/trans) | octanone | octanol |
| Before | 58.1 | 3.1 | 2.7 | 3.5 | 32.6 |
| distillation | | | | | |
| Fraction 1 | 94.2 | 3.1 | 2.5 | 0.0 | 0.2 |
| Fraction 2 | 93.2 | 3.5 | 2.9 | 0.1 | 0.3 |
| Fraction 3 | 93.1 | 3.6 | 3.0 | 0.1 | 0.2 |
| Fraction 4 | 92.7 | 3.9 | 3.2 | 0.0 | 0.2 |
| Fraction 5 | 91.7 | 4.3 | 3.6 | 0.1 | 0.3 |
| Fraction 6 | 91.3 | 4.4 | 3.8 | 0.1 | 0.4 |
| Fraction 7 | 89.6 | 5.1 | 4.6 | 0.5 | 0.2 |
| Fraction 8 | 88.6 | 5.7 | 5.2 | 0.1 | 0.4 |
| Fraction 9 | 85.4 | 7.1 | 6.9 | 0.2 | 0.4 |
| Fraction 10 | 55.1 | 7.3 | 7.9 | 9.8 | 19.9 |
| Fraction 11 | 2.0 | 0.3 | 0.3 | 28.8 | 68.6 |
| Fraction 12 | 0.0 | 0.0 | 0.0 | 5.8 | 94.2 |
| Fraction 13 | 0.1 | 0.0 | 0.0 | 0.4 | 99.5 |

Formic acid (103 g) was added with stirring to distilled 1-octene (100 g, 91,1 area-%). The solution was heated to 50 °C. Hydrogen peroxide (50%, 82 g) was added slowly, keeping the reaction temperature below 60 °C. After addition, the reaction mixture was stirred to allow full oxidation and destruction of excess peroxide. The phases were separated, and the organic phase was washed with water. Sodium hydroxide (50% aqueous solution) was slowly added to the organic phase, keeping the temperature below 60 °C, until the pH of the mixture is stabilized between 8 and 9. After phase separation and a second washing with water, the crude product was dried in vacuo at 60 °C. The crude 1,2-octanediol was fractionally distilled in vacuo over a 300 mm Vigreux column (bp 92 °C/2 mbar). Purified 1,2-octanediol (88.7 g) was obtained. The purity of the collected fractions is summarized in Table 12.

TABLE 12

| Fraction | Mass (g) | 1,2-octanediol (area-%) |
|---|---|---|
| 1 | 19.4 | 76.9 |
| 2 | 19.0 | 90.3 |
| 3 | 20.7 | 96.7 |
| 4 | 18.5 | 99.6 |
| 5 | 11.1 | 99.5 |

The results obtained show that it is possible to purify the 1,2-alkanediol by removing the isomeric straight-chain vicinal alkanediols by fractional distillation.

The present invention is in no way limited to the embodiments described in the examples and/or shown in the figures. On the contrary, methods according to the present invention may be realized in many different ways without departing from the scope of the invention.

1. A method for the preparation of primary alkenes from secondary alcohols, comprising:
dehydrating said secondary alcohols in the presence of a zirconia (ZrO₂) catalyst to produce primary alkenes;
characterized in that, the zirconia catalyst used in the dehydrating contains less than 0.2 wt. % silicon dioxide by weight of the zirconia catalyst and has a surface area of less than 100 m²/g.

2. The method according to claim 1, wherein the secondary alcohols are derived from bio-based secondary alcohols derived from a renewable resource.

3. The method according to claim 2, wherein the renewable resource is castor oil, cellulose or hemicellulose.

4. The method according to claim 1, wherein the dehydrating is carried out in the presence of an inert carrier gas with a flow rate of between 150 and 5000 liters of inert carrier gas (measured at standard conditions) per liter of secondary alcohol used.

5. The method according to claim 1, wherein the secondary alcohols are straight chain, branched chain or cyclic secondary alcohols having from 2 to 30 carbon atoms.

6. The method according to claim 1, wherein the secondary alcohols are 3-methyl-2-butanol, 2-pentanol, 2-hexanol, 2-heptanol, 2-octanol, 2-nonanol, 2-decanol, 2-undecanol, 2-dodecanol, or any combination thereof.

7. The method according to claim 1, wherein the zirconia catalyst is present in the form of pellets with an average diameter of between 0.1 mm and 10 mm.

8. The method according to claim 1, wherein the zirconia catalyst contains at least 80% of zirconia in the monoclinic phase.

9. The method according to claim 1, wherein the dehydrating is carried out as a continuous gas phase reaction.

10. The method according to claim 9, wherein secondary alcohols are fed to a reactor with a liquid hourly space velocity (LHSV) of between 0.1 and 10 h⁻¹.

11. The method according to claim 1, wherein the zirconium catalyst has a surface area of less than 40 m²/g.

12. The method according to claim 1, wherein the dehydrating is carried out at a temperature between 200 °C and 500 °C.

13. The method according to claim 1, wherein the catalyst is preheated at a temperature between 200 °C and 400 °C under an inert gas stream.

14. The method according to claim 1, wherein the dehydrating is carried out in a continuous fluidized bed reactor.

15. The method according to claim 1, wherein the dehydrating is carried out in a continuous fluidized bed reactor, and wherein the zirconium catalyst has a surface area of less than 40 m²/g.

16. A method for the preparation of 1,2-alkanediols from secondary alcohols, comprising:
preparing primary alkenes from secondary alcohols according to claim 1; and
oxidizing said primary alkenes to form 1,2-alkanediols.

17. The method according to claim 16, further comprising a distillation step between the dehydrating and oxidizing.

18. The method according to claim 1, wherein the dehydrating is carried out in the presence of an inert carrier gas with a flow rate of between 1500 and 2500 liters of inert carrier gas (measured at standard conditions) per liter of secondary alcohol used.

19. The method according to claim 1, wherein the zirconia catalyst is present in the form of pellets with an average diameter of between 1 mm and 7 mm.

20. The method according to claim 1, wherein the zirconia catalyst contains at least 95% of zirconia in the monoclinic phase.
